(19) United States
(12) Patent Application Publication
(10) Pub. No.: US 2013/0243706 A1
(43) Pub. Date: Sep. 19, 2013

(54) TRANSFER RESISTANT COSMETICS AND METHOD OF MAKING

(71) Applicants: Salvatore J. Barone, Staten Island, NY (US); Lethu Nguyen, Colonia, NJ (US); Fredericke Vance Maillard, Paris (FR)

(72) Inventors: Salvatore J. Barone, Staten Island, NY (US); Lethu Nguyen, Colonia, NJ (US); Fredericke Vance Maillard, Paris (FR)

(73) Assignee: Coty Inc., New York, NY (US)

(21) Appl. No.: 13/822,568

(22) PCT Filed: Nov. 8, 2011
(86) PCT No.: PCT/US11/59744

§ 371 Date: May 29, 2013

Related U.S. Application Data
(60) Provisional application No. 61/411,172, filed on Nov. 8, 2010.

(51) Int. Cl. A61K 8/92; A61Q 3/00; A61K 8/31; A61Q 15/00; A61K 8/81; A61Q 19/00; A61Q 1/00

(52) U.S. Cl. CPC A61K 8/922; A61Q 19/00; A61Q 3/00; A61Q 1/00; A61Q 15/00; A61K 8/8111; A61K 8/31
USPC 424/59; 514/772; 424/70.11; 424/61; 424/65

(57) ABSTRACT

Embodiments claimed and claimed herein include anhydrous cosmetic composition embodiments. The anhydrous cosmetic composition embodiments include coconut alkanes comprising a volatile oily composition having from 50 to 100% by weight of a mixture of linear paraffins that include 70 to 99% by weight of at least one linear paraffin selected from C %, C₁₀, C₁₂ paraffins and mixtures thereof. The composition also includes a coconut gel comprising vegetable oil, Cocos Nucifera Oil, a styrene/butadiene copolymer and polyethylene. The composition embodiments also include polyethylene, caprylyl glycol, and a mixture of Acacia Decurrens/Jojoba/Sunflower Seed wax/Polyglyceryl 3-ester in concentrations effective for ensuring coconut alkanes are melted and homogeneous; and one or more anhydrous actives.

### CLAIM OF PRIORITY

This patent application claims the benefit of priority of U.S. Provisional Patent Application Ser. No. 61/411,172, entitled TRANSFER RESISTANT COSMETICS AND METHOD OF MAKING, which was filed on Nov. 8, 2010, and which is hereby incorporated by reference herein in its entirety.

### FIELD

Inventive subject matter disclosed herein relates to anhydrous cosmetic embodiments and method embodiments for making anhydrous cosmetics having transfer resistant properties.

### BACKGROUND

Use of cosmetics has had a long and interesting history. Women started coloring their lips at least about 5000 years ago when Mesopotamian women decorated their lips with crushed jewels. Ancient Egyptians applied minerals to their faces to define their eyes and other features. Roman women treated their teeth and eyebrows.

For many centuries, men and women used a whitening agent on their faces. The whitening agent included one or more carbonates, hydroxides and lead oxide. This unfortunate agent was replaced in the nineteenth century by zinc oxide.

Modern cosmetics have ranges of purpose and effectiveness not contemplated by earlier generations. Purposes are both aesthetic and are related to improvement or maintenance of skin condition.

### SUMMARY

Embodiments claimed and claimed herein include anhydrous cosmetic composition embodiments. The anhydrous cosmetic composition embodiments include coconut alkanes comprising a volatile oily composition having from 50 to 100% by weight of a mixture of linear paraffins that include 70 to 99% by weight of at least one linear paraffin selected from C %, C₁₀, C₁₂ paraffins and mixtures thereof. The composition also includes a coconut gel comprising vegetable oil, Cocos Nucifera Oil, a styrene/butadiene copolymer and polyethylene. The composition embodiments also include polyethylene, caprylyl glycol, and a mixture of Acacia Decurrens/Jojoba/Sunflower Seed wax/Polyglyceryl 3-ester in concentrations effective for ensuring coconut alkanes are melted and homogeneous. The composition also includes one or more anhydrous actives.

Inventive subject matter also includes a method for imparting transfer resistance to a cosmetic, comprising: adding to one or more actives, a coconut alkane mixture comprising a volatile oily composition having from 50 to 100% by weight of a mixture of linear paraffins that include 70 to 99% by weight of at least one linear paraffin selected from C %, C₁₀, C₁₂ paraffins and mixtures thereof; heating a coconut gel comprising vegetable oil, Cocos Nucifera Oil, a styrene/butadiene copolymer and polyethylene; and adding the one or more actives and coconut alkane mixture and coconut gel together.

Inventive embodiments also include a method for making a cosmetic having transfer resistance, comprising: combining one or more dry actives with an amount of coconut alkanes comprising a volatile oily composition having from 50 to 100% by weight of a mixture of linear paraffins that include 70 to 99% by weight of at least one linear paraffin selected from C %, C₁₀, C₁₂ paraffins and mixtures thereof effective to form a paste; heating a mixture of a coconut gel comprising vegetable oil, Cocos Nucifera Oil, a styrene/butadiene copolymer and polyethylene to a temperature of about 85 to 90 degrees Centigrade; heating coconut alkanes comprising a volatile oily composition having from 50 to 100% by weight of a mixture of linear paraffins that include 70 to 99% by weight of at least one linear paraffin selected from C %, C₁₀, C₁₂ paraffins and mixtures thereof; adding the heated coconut gel to the heated coconut alkanes; adding the paste to the mixture of coconut gel and coconut alkanes; and optionally adding fragrance.

Inventive subject matter also includes a skin care composition comprising vegetable oil, cocos nucifera, coconut, oil and styrenelbutadiene copolymer, coconut alkanes and one or more actives effective for treating skin.

Inventive embodiments further include a scalp care or hair composition comprising vegetable oil, cocos nucifera, coconut, oil and styrene/butadiene copolymer and coconut alkanes and one or more actives effective for treating scalp care or hair.

Inventive embodiments also include an eyelash composition comprising vegetable oil, cocos nucifera, coconut, oil and styrene/butadiene copolymer and coconut alkanes and one or more actives effective for treating eyelashes

Inventive embodiments further include a nail composition comprising vegetable oil, cocos nucifera, coconut, oil and styrene/butadiene copolymer and coconut alkanes and one or more actives effective for treating a mammalian

In the Figures

FIG. 1 illustrates a Lipstick Transfer Scale embodiment.

### DETAILED DESCRIPTION

The following detailed description includes references to embodiments, which are described in enough detail to enable those skilled in the art to practice the invention. The embodiments may be combined, other embodiments may be utilized, or structural, and logical changes may be made without departing from the scope of the present invention. The following detailed description is, therefore, not to be taken in a limiting sense, and the scope of the inventive subject matter is defined by the appended claims and their equivalents.

In this document, the terms “a” or “an” are used to include one or more than one and the term “or” is used to refer to a nonexclusive “or” unless otherwise indicated. In addition, it is to be understood that the phraseology or terminology employed herein, and not otherwise defined, is for the purpose of description only and not of limitation. Furthermore, all publications, patents, and patent documents referred to in this document are incorporated by reference herein in their entirety, as though individually incorporated by reference. In the event of inconsistent usages between this document and those documents so incorporated by reference, the usage in the incorporated reference should be considered supplementary to that of this document; for irreconcilable inconsistencies, the usage in this document controls.

Embodiments disclosed herein include a cosmetic with long lasting transfer resistance. The cosmetic composition embodiments include a gel having a vegetable oil, coconut oil and a styrene/butadiene copolymer. One gel product that includes these ingredients is NatureVgel, manufactured by AppleChem, of Newark, N.J. The long lasting transfer resistant cosmetic composition also includes coconut alkanes, provided in one embodiment, by a product called. Vegelight 1214C, manufactured by BioSyntheIs, of St. Cyr Sous Dourdan, France. It is believed that the combination of the gel that includes vegetable oil, coconut oil, styrenelbutadiene copolymer, and coconut alkanes imparts to the cosmetic embodiments long lasting transfer resistance. The cosmetic embodiments described herein are manufactured in conventional forms, such as sticks, liquids, and powders. For some embodiments, the inventive powder embodiments include particles or flakes or beads or twills. The particle size may be millimeter-sized for some embodiments or micron sized for other embodiments. For some embodiments, the inventive cosmetic powder includes particles sized for spraying. For some embodiments, the inventive particles are prepared with a medium to form a paste.

The term “stick” as used herein, refers to cosmetic compositions molded into the form of a stick. For some embodiments, the compositions are heated until molten and then poured into a mold and cooled. Stick embodiments also include anhydrous compositions capable of being formed into sticks.

One cosmetic formulation embodiment is directed to a lipstick having shiny, long lasting transfer resistance. The lipstick cosmetic formulation is as follows

| # | INCI Name | Raw Material Name | % weight |
|---|---|---|---|
| Phase A | | | |
| 1 | Vegetable Oil, Cocos | NatureVgel - | 15.00 |
| | Nucifera(coconut)Oil and | Fractionated coconut | |
| | Styrene/Butadien Copolymer | | |
| 2 | Coconut Alkanes | Vegelight 1214C | 41.12 |
| Phase B | | | |
| 3 | Polyethylene | Performalene 400 | 15.00 |
| Phase C | | | |
| 4 | Acacia Decurrens/Jojoba/ | Hydracire S | 5.00 |
| | Sunflower Seed wax/ | | |
| | Polyglyceryl 3-ester | | |
| 5 | Caprylyl Glycol | Lexgard O | 0.50 |
| Phase D | | | |
| 6 | D&C Red#7 Calcium Lake | Red#7 C19-011 | 0.86 |
| 7 | IronOxides | Red Oxide C33-2199 | 1.90 |
| 8 | IronOxides | Black Oxide 300-401 | 0.79 |
| 9 | TITANIUM DIOXIDE | TiO2 C47-056 | 1.90 |
| 10 | Coconut Alkanes | Vegelight 1214C | 5.000 |
| Phase E | | | |
| 11 | Mica and Lauroyl Lysine | Mica with Lauroyl | 4.000 |
| | | Lysine | |
| 12 | TITANIUM DIOXIIH(and) | TIMIRON MP-115 | 7.00 |
| | MICA | | |
| 13 | MICA | Silk Mica | 1.53 |
| Phase F | | | |
| 14 | Fragrance | Fleuri | 0.40 |
| | TOTALS: | TOTALS: | 100.00 |

Ingredients of the cosmetic formulation having long lasting transfer resistance include the following:

### Gel

Gel embodiments include a blend of from 1 to 50 weight percent of at least one block copolymer, wherein the block copolymer has at least one polystyrene block and at least one unsaturated rubber block; and from 99 to 50 weight percent of natural oil. The composition results in a clear natural oil gel.

The block copolymer portion represents from 1 to 50% by weight of the composition. For some embodiments, the block copolymer is a single block copolymer or a mixture of different block copolymers. The block copolymers have unsaturated rubber blocks, for examples, SBS, SB, SIS, SI, (SB)n, and (SI)n. The block copolymers have a polystyrene content from 5% to 80%, for some embodiments from 10% to 50%, and for some embodiments, from 15% to 40% by weight of the block copolymer. Examples of these copolymers include Kraton® D Polymers and Vector® polymers.

The natural oils of the gel described herein may be plant or animal oils, and tend to be liquid at the room temperature. They are extracted from a wide range of plants and animals. An example of animal oils is fish oil. The most preferred natural oils of the invention are plant or vegetable oils. For example a preferred vegetable oil is jojoba oil, which is a monoester of eicosanoic and docosanoic acids and eicosanol and docosanol alcohols. Most other vegetable oils are triglycerides of glycerin and fatty acids which are largely unsaturated fatty acids such as oleic acid and linoleic acid. The vegetable oils of the present invention include non-genetically modified oils, genetically modified oils, organic grown oils, non-organic grown oils, and mixtures thereof.

In general, vegetable oils are readily available and prepared by extraction from plant seeds. Examples of the vegetable oils or plant oils include, but are not limited to, almond, avocado, castor, coconut, corn, cottonseed, olive, peanut, rice bran, safflower, sesame, soybean, sunflower, walnut, canola, refined palm, meadaowfoam, tea tree oil, etc.

The clear natural oil gels used in inventive cosmetic composition embodiments described herein may additionally contain auxiliary ingredients including a) skin compatible hydrophobic emollients including, but not limited to, sensory enhancers, synthetic esters, and hydrocarbon oils, the preferred ones being those derived from natural oils; b) skin benefit agents; and c) colorants, fumed silica, cornstarch, antioxidants, etc. Additional information regarding the get is found in U.S. Pat. No. 7,674,848, which is herein incorporated by reference.

### Coconut Alkanes:

The coconut alkanes include a volatile oily composition having from 50 to 100% by weight of a mixture of linear paraffins that include 70 to 99% by weight of at least one linear paraffin selected from C %, C₁₀, C₁₂ paraffins and mixtures thereof, (ii) 1 to 30% by weight of at least one C₁₄ to C₂₄ linear paraffin, and (b) from 0 to 50% by weight of at least one non-volatile oil.

As used herein, the word, “between” refers to including the cited limits.

The coconut alkanes have a volatility within the same range as cycllomethicones, i.e. a vapour pressure in the region of 0.001 to 300 mm Hg at ambient temperature (20° C.) and atmospheric pressure. The coconut alkanes form a film having a soft, non-greasy touch and a reduced gloss similar to those of cyclomethicones. This oily composition further has a flash point (measured as per the ASTM D93 standard) below 100° C., and/or a kinematic viscosity below 5 cSt, or between 1 and 3 cSt, at 40° C. This association of raw materials may thus partly or completely substitute the volatile silicones conventionally used in cosmetic compositions and more particularly cyclopentasiloxane and mixtures containing same (such as DC345® sold by DOW CORNING). The paraffins (or fatty alkanes) contained in the oily composition according to the invention may be advantageously obtained according to a method comprising the following successive steps:

1-dehydration of at least one Ce—C₂₄ fatty alcohol to obtain an alkene, and

2-hydrogenation of said alkene to an alkane. The first step of this method may particularly be implemented as described in the document US 2008/0287722, i.e. at a temperature of 190 to 260° C., for some embodiments, temperatures of 220 to 250° C., in the presence of a dehydration catalyst consisting of trifluoromethane sulphonic acid, which may represent 0.5 to 3% of the weight of the alcohol, for example. The alcohol dehydrated in this step may be obtained from plant sources and particularly be obtained by saponification of natural oils or fats. However, it is preferable for it to be obtained according to a method comprising a transesterification step of fatty acid triglycerides, for some embodiments, of plant origin, followed by a hydrogenation step of the fatty acid esters (for example methyl esters) obtained. The use of fatty alcohols of plant origin leads to alkenes containing an even number of carbon atoms, generally in mixture form. This mixture may also comprise a minor amount of branched alkenes.

The second step of this method may be implemented in a conventional manner for those skilled in the art, according to techniques used in the food processing industry for hydrogenating oils, and particularly placing the alkene(s) in contact with a catalyst comprising a transition metal. The alkanes obtained preferably contain, as above, an even number of carbon atoms. They are linear, although they may also include a minor amount of branched alkanes.

Obviously, this method may further comprise other steps (preliminary, intermediate and/or subsequent steps) than those mentioned above. Alternatively, commercially available paraffins, such as those available from SASOL under the brand name Parafol® (particularly Parafol® 14-97 for tetradecane) may be used in the oily composition according to the invention. The oily composition according to the invention comprises 70 to 99% by weight, for example 70 to 90% or 90 to 99% by weight, Cg, C₁₀ and/or C₁₂ paraffin(s) and 1 to 30% by weight, for example 10 to 30% or 1 to 10% by weight, C₁₄ to C₂₄ paraffin(s). These two types of paraffins may be obtained separately and mixed, or obtained jointly from a mixture of fatty alcohols, particularly of plant origin, according to the method described above. Among the C %, C₁₀ and/or C₁₂ paraffin(s), the C₁₂ paraffin is preferred. Moreover, as explained above, the C₁₄ to C₂₄ paraffin(s) are preferably chosen among those having an even number of carbon atoms, still preferably the C₁₄ paraffin. Furthermore, preferably, the oily composition according to the invention comprises, or consists only of, dodecane and tetradecane.

In addition to the abovementioned paraffins, and according to the desired volatility, the oily composition according to the invention may contain at least one non-volatile oil. According to the present invention, the term “oil” refers to a liquid compound at ambient temperature (25° C.), which, when introduced at a rate of at least 1% by weight in water at 25° C., is not at all soluble in water, or soluble at a rate of less than 10% by weight, with reference to the weight of oil introduced into the water. In this description, the term “non-volatile oil” refers to an oil remaining on the skin at ambient temperature and atmospheric pressure for a plurality of hours, in the absence of friction, and/or having a vapour pressure less than 0.001 mm Hg under these conditions.

Examples of non-volatile oils include: mineral or synthetic branched hydrocarbons, synthetic (poly)esters and(poly)others and particularly (poly)esters C₂-C₂₄ (such as C₆-C₂₀) acids and C₂-C₂₄ (such as C₆-C₂₀) alcohols or polyols, which are advantageously branched, C_{O}-C₂₀ fatty acid triglycerides, vegetable dialkyl carbonates such as dicaprylyl carbonate, branched and/or unsaturated fatty acids (such as linoteic and linolenic acids), branched and/or unsaturated fatty alcohols (such as octyidodecanol or hexyldecanol), silicone fluorosilicone oils, fluorinated oils, and mixtures thereof. The term “hydrocarbon” refers to an oil containing only hydrogen and carbon atoms. Examples of non-volatile hydrocarbon oils are polybutene, hydrogenated polyisobutene, polydecene, hydrogenated polydecene, squalane, non-volatile paraffin oils and mixtures thereof. The(poly)esters of C₂-₂₄ acids and C₀-C₂₀ alcohols and polyols, which represent the preferred category of non-volatile oils according to the invention, particularly include mono- and diesters such as ethyl acetate, isopropyl acetate, oleyl acetate, isononyl isononanoate, ethylhexyl isononanoate, hexyl neopentanoate, ethylhexyl neopentanoate, isodecyl neopentanoate, isostearyl neopentanoate, heptyl undecylenate, neopentyiglycol diheptanoate, neopentyiglycol diethylhexanoate, pentaerythrityl tetraethythexanoate, propanediol dicaprylate, neopentylglycol dicaprylate/dicaprate, isopropyl myristate, isopropyl palmitate, hexyl laurate, the mixture of coco caprate and caprylate, C₁₂ C₁₅ alcohol benzoates, and mixtures thereof.

Examples of vegetable oils are in particular wheat germ, sunflower, grape seed, sesame, corn, apricot, castor, shea, avocado, olive, soya, sweet almond, palm, rapeseed, cotton, hazelnut, macadamia, jojoba, alfalfa, poppy seed, pumpkin seed, sesame, marrow, rapeseed, blackcurrant, evening primrose, millet, barley, quinoa, safflower, candlenut, passiflora, musk rose or camellia oils.

The term “silicone oil” refers to an oil comprising at least one silicon atom, and particularly at least one Si—O group. Non-volatile silicon oils particularly include polydimethylsiloxanes containing at least 8 silicon atoms, polyaikyiniethylsiloxanes in which the alkyl chain contains 8 to 20 carbon atoms and oils identified using the INCI name phenyl trimethicone.

The oily composition according to the invention contains 50 to 100% by weight of paraffin mixture and 0 to 50% by weight of non-volatile oil(s), for example 70 to 95% by weight, for some embodiments, 85 to 95% by weight, of paraffin mixture and 5 to 30% by weight, for some embodiments, 5 to 15% by weight, of non-volatile oil(s). Oily compositions according to this invention are also marketed by One coconut alkane formulation is sold by BIOSYNTHIS under the trade name Vegelight®. 1214.

The oily composition described above is advantageously intended for use in the formulation of a cosmetic.

### Pigments and Powders

Some cosmetic composition embodiments also include one or more pigments and powders in a concentration range of 5-50% by weight of the total composition Particle size of the pigments and powders range from 0.02 to 200 microns, but may be larger or smaller for other embodiments. Suitable powders include bismuth oxychloride, titanated mica, fumed silica, spherical silica, polymethylmethacrylate, micronized teflon, boron nitride, acrylate copolymers, aluminum silicate, aluminum starch octenylsuccinate, bentonite, calcium silicate, cellulose, chalk, corn starch, diatomaceous earth, fuller's earth, glyceryl starch, hectorite, hydrated silica, kaolin, magnesium aluminum silicate, magnesium maltodextrin, montmorillonite, microcrystalline cellulose, rice starch, silica, talc, mica, titanium dioxide, zinc laurate, zinc myristate, zinc rosinate, alumina, attapulgite, calcium carbonate, calcium silicate, dextran, kaolin, nylon, silica silylate, silk powder, sericite, soy flour, tin oxide, titanium hydroxide, trimagnesium phosphate, walnut shell powder, or mixtures thereof. The powders described herein may be surface treated with lecithin, amino acids, mineral oil, silicone oil or various other agents either alone or in combination, which coat the powder surface and render the particles more lipophilic in nature.

Some powder embodiments include organic and inorganic pigments. The organic pigments include azo, indigold, triphenylmethane, anthraquinone, and xanthine dyes which are designated as D &C and FD &C blues, browns, greens, oranges, reds, yellows, etc. Organic pigments include insoluble metallic salts of certified color additives, referred to as the Lakes. Inorganic pigments include iron oxides, ultramarines, chromium, chromium hydroxide colors, and mixtures thereof. Some composition embodiments include both pigment and non-pigmented powders. The weight ratio of pigment to non-pigmented powder will range from 1:20 to 20:1.

### Fragrances

Embodiments disclosed herein are either fragrance free or include a fragrance compatible with a cosmetic. In one embodiment, the fragrance is Fleuri.

In one embodiment, the cosmetic composition is a lipstick of high transfer resistance. The lipstick is made with a three-roller milt a stirrer and a propeller. One step of the method includes preparing a colorant phase. The colorant phase includes combining colorant ingredients such as D&C Red NO. 7 Calcium Lake, iron oxides, titanium dioxide, and other colorants as desired. Coconut alkanes, such as Vegelight 1214C, are added to the colorant ingredients in a concentration effective to make a paste. The paste is passed through a 3-roller at least 3-4 times. The pigment grind is checked under a Hegman gauge to ensure good pigment dispersion.

In another step, a coconut gel, such as Nature Vgel, is added to a container, such as a reactor. The coconut gel is heated to 85 to 90° C. The coconut gel is mixed to obtain a uniform mixture. Polyethylene is added to the heated coconut gel at a temperature of 85 to 90° C. to form a PE-gel mixture. The PE-gel mixture is then well mixed to ensure that waxes are completely melted or homogeneous.

Coconut alkanes, for some embodiments, Vegelight 1214C are added to the coconut gel and polyethylene mixture at a temperature of 85° C., to 90° C., forming a PE-gel-alkane mixture. The PE-gel-alkane mixture then mixed.

Acacia Decurrens/Jojoba/Sunflower Seed wax/Polyglyeryl 3-ester is added to the PE-gel-alkane mixture. Caprylyl glycol is also added to the coconut alkanes, gel mixture.

The colorant paste, including the coconut alkanes, is quickly added to the heated mixture of coconut gel and polyethylene to form anew mixture. The new mixture is mixed and held at a temperature within a range of 85 to 90° C.

The temperature of 85° C. to 90° C. is maintained, Mica and lauroyl lysine, titanium dioxide and mica and mica are added one at a time to the new mixture.

For some embodiments, a fragrance is added to the mixture and mixed. The mixture is then poured into stick molds at 85° C. to make lipstick.

Cosmetic compositions that include the gel that includes NatureVgel-Fractionated coconut oil and coconut alkanes having a volatile oily composition having from 50 to 100% by weight of a mixture of linear paraffins that include 70 to 99% by weight of at least one linear paraffin selected from C %, C₁₀, C₁₂ paraffins and mixtures thereof; a coconut gel comprising vegetable oil, Cocos Nucifera Oil, a styrene/butadiene copolymer and polyethylene; polyethylene, caprylyl glycol, and a mixture of Acacia Decurrens/Jojoba/Sunflower Seed wax/Polyglyceryl 3-ester in concentrations effective for ensuring coconut alkanes are melted and homogeneous; and one or more anhydrous actives are usable in a variety of anhydrous cosmetics, in addition to lipstick.

The anhydrous cosmetics include skin care compositions, scalp care compositions, and compositions for hair, eyelashes and nails. The anhydrous cosmetics disclosed herein have an improved transfer resistance compared to cosmetics that do not include the coconut gel, NatureVgel-Fractionated coconut oil and coconut alkanes.

Skin care and other cosmetic anhydrous actives include but are not limited to petroleum-based emollients, vegetable oils, hydrogenated vegetable oils, and their derivatives; branched hydrocarbons; fatty alcohol ethers; free sterols, sterol esters and their derivatives; sphingolipids; phospholipids; and mixtures thereof. Suitable petroleum-based emollient include petrolatums, i.e., hydrocarbons or mixtures of hydrocarbons; particularly preferred are hydrocarbons having chain lengths of from C10 to C100. Petroleum-based emollients within this chain length range include mineral oil and petrolatum. Mineral oil usually refers to less viscous mixtures of hydrocarbons having from 10 to 30 carbon atoms, though the hydrocarbon molecular weight distribution may vary. Since the lower molecular weight hydrocarbons can cause irritation in some individuals, mineral oils having a small percentage of lower molecular weight hydrocarbons are preferred. Petrolatum usually refers to more viscous mixtures of hydrocarbons of higher molecular weight hydrocarbons.

Skin care actives usable in cosmetic formulations described herein include, but are not limited to, acerola, ascorbic acid, algae extract, seaweed, Lithotanmium calcarum seaweeds, micronized laminaria digitata, and micronized focus vesiculosus, alginate, allantion, aloe vera, amino acids, apricot seed oil, arbutin, arnica extract, avocado oil, barley extract, bearberry extract, bentone white clay, shea butter, tocopherol, tea tree oil, hydroquinone, emu oil, hyaluronic acid, peptides, collagen, green tea, and argireline.

Actives for mascara composition embodiments include but are not limited to one or more of the following: beeswax, carnauba wax, paraffin, mineral oil, lanolin, linseed oil, castor oil, oil of turpentine, eucalyptus oil, sesame oil, stearic acid, stiffeners such as ceresin and gums such as gum tragacanth and methyl cellulose. Some mascaras include fibers, such as rayon fibers, which make the product more viscous.

One cosmetic embodiment relates to a two-part cosmetic that imparts a filling and/or swelling effect to hair, eyebrows or eyelashes. The formulation uses an anhydrous base coat with a water absorbing ingredient such as a salt of acrylic or polyacrylic acid and a carbon dioxide releasing compound which is a carbonate or bicarbonate to expand or swell the film former of the top coat with the in situ generated carbon dioxide when the water containing top coat is applied to the anhydrous base coat. The coconut alkanes and coconut gel described herein are added to the anhydrous base coat and impart an improved transfer resistance to the cosmetic compared to a cosmetic without the coconut alkanes and coconut gel. Additional information concerning this formulation is disclosed in WO2009/043898, which is herein incorporated by reference.

Another cosmetic composition embodiment into which the coconut alkanes and coconut gel can be added includes deodorant and antiperspirant embodiments. The cosmetic compositions of deodorants and antiperspirants include from about 0.01% to about 70%, of one or more cosmetic actives. Exemplary cosmetic actives include moisturizers, emollients, fillers, colorants, perfumes or fragrances, skin conditioners, vitamins, photoprotectants (e. g., sunscreens), antiperspirants, antioxidants, anti-wrinkle materials, as well as any other materials suitable for topical applications. Antiperspirant and deodorant composition embodiments effective for receiving the coconut alkanes and coconut gel are disclosed in U.S. Pat. No. 7,041,278; U.S. Pat. No. 6,358,498; and US Pub. No. 20090220555A1, which are herein incorporated by reference. The coconut alkanes and coconut gel improve the transfer resistance of the deodorant and antiperspirant composition when applied to skin.

The transfer resistance of one cosmetic, a lipstick made with the formulation disclosed in the table herein was evaluated. In particular, panelists were given a blinded lipstick sample and applied an even coat of the lipstick and let it dry for fifteen minutes. All panelists were women above the age of eighteen years. Each panelist was given a white tile to kiss using moderate pressure. Three expert evaluators scored the amount of transfer according to the Lipstick Transfer Scale, as illustrated in FIG. 1.

No adverse reactions were reported. Expert evaluator scores are shown in the table below. To be considered a transfer-resistant lipstick, a lipstick must have an average score of 1.36.

| Evaluator 1 | Evaluator 2 | Evaluator 3 | Average | Panelist |
|---|---|---|---|---|
| 1 | 1 | 1 | 1 | 1.00 |
| 2 | 2 | 3 | 2.5 | 2.50 |
| 3 | 1 | 2.5 | 1.5 | 1.67 |
| 4 | 1 | 1 | 1 | 1 |
| 5 | 1.5 | 2 | 1.5 | 1.67 |
| 7 | 1 | 1 | 1 | 1.00 |
| 8 | 0.5 | 1 | 0.5 | 0.67 |

Mean 1.36

The embodiments are described in sufficient detail to enable those skilled in the art to practice the invention. Other embodiments may be utilized and formulation and method of using changes may be made without departing from the scope of the invention. The detailed description is not to be taken in a limiting sense, and the scope of the invention is defined only by the appended claims, along with the full scope of equivalents to which such claims are entitled.

It will be appreciated by those skilled in the art that changes could be made to the embodiments described above without departing from the broad inventive concept thereof. It is understood, therefore, that this invention is not limited to the particular embodiments disclosed, but it is intended to cover modifications within the spirit and scope of the present invention as defined by the present description

What is claimed is:

1. An anhydrous cosmetic comprising:
Coconut alkanes comprising a volatile oily composition having from 50 to 100% by weight of a mixture of linear paraffins that include 70 to 99% by weight of at least one linear paraffin selected from C %, C₁₀, C₁₂ paraffins and mixtures thereof;
a coconut gel comprising vegetable oil, Cocos Nucifera Oil, a styrene/butadiene copolymer and polyethylene;
polyethylene, caprylyl glycol, and a mixture of Acacia Decurrens/Jojoba/Sunflower Seed wax/Polyglyceryl 3-ester in concentrations effective for ensuring coconut alkanes are melted and homogeneous; and
one or more anhydrous actives.

2. The anhydrous cosmetic of claim 1, wherein the one or more actives comprise a colorant paste.

3. The anhydrous cosmetic of claim 1, wherein the anhydrous cosmetic is a skin care composition.

4. The anhydrous cosmetic of claim 1, wherein the anhydrous cosmetic is a scalp care composition.

5. The anhydrous cosmetic of claim 1, wherein the anhydrous cosmetic is employed in compositions for one or more of hair, eyelashes and nails.

6. A method for imparting transfer resistance to a cosmetic, comprising:
adding to one or more actives, a coconut alkane mixture comprising a volatile oily composition having from 50 to 100% by weight of a mixture of linear paraffins that include 70 to 99% by weight of at least one linear paraffin selected from C %, C₁₀, C₁₂ paraffins and mixtures thereof;
heating a coconut gel comprising vegetable oil, Cocos Nucifera Oil, a styrene/butadiene copolymer and polyethylene; and
adding the one or more actives and coconut alkane mixture and coconut gel together.

7. The method of claim 6, wherein the coconut gel is heated to a temperature within a range of 85 degrees C. to 90 degrees C.

8. The method of claim 6, further comprising adding the one or more actives and coconut gel and coconut alkane mixture to a mold at a temperature of 85 degrees C.

9. A method for making a cosmetic having transfer resistance, comprising:
combining one or more dry actives with an amount of coconut alkanes comprising a volatile oily composition having from 50 to 100% by weight of a mixture of linear paraffins that include 70 to 99% by weight of at least one linear paraffin selected from C %, C₁₀, C₁₂ paraffins and mixtures thereof effective to form a paste;
heating a mixture of a coconut gel comprising vegetable oil, Cocos Nucifera Oil, a styrene/butadiene copolymer and polyethylene to a temperature of about 85 to 90 degrees Centigrade;
heating coconut alkanes comprising a volatile oily composition having from 50 to 100% by weight of a mixture of linear paraffins that include 70 to 99% by weight of at least one linear paraffin selected from C %, C₁₀, C₁₂ paraffins and mixtures thereof;
adding the heated coconut gel to the heated coconut alkanes;
add the paste to the mixture of coconut gel and coconut alkanes; and
optionally add fragrance.

10. The method of claim 9, wherein the heated mixture of coconut gel and heated coconut alkanes are poured into a mold.

11. The method of claim 9, wherein the heated mixture of coconut gel and heated coconut alkanes is cooled, solidified and particulated to make a powder.

12. A skin care composition or sunscreen composition comprising vegetable oil cocos nucifera, coconut, oil and styrene/butadiene copolymer and coconut alkanes.

13. A scalp care or hair composition comprising vegetable oil, cocos nucifera, coconut, oil and styrene/butadiene copolymer and coconut alkanes.

14. An eyelash composition comprising vegetable oil, cocos nucifera, coconut, oil and styrene/butadiene copolymer and coconut alkanes.

15. A nail composition comprising vegetable oil, cocos nucifera, coconut, oil and styrene/butadiene copolymer and coconut alkanes.

16. An antiperspirant or deodorant composition comprising vegetable cocos nucifera, coconut, oil and styrene/butadiene copolymer and coconut alkanes.
